(19) United States
(12) Patent Application Publication
(10) Pub. No.: US 2010/0305195 A1
(43) Pub. Date: Dec. 2, 2010

(54) MICRORNA MEDIATOR OF CARDIOMYOPATHY AND HEART FAILURE

(75) Inventor: Patrick K. Umeda, Birmingham, AL (US)

Correspondence Address: FISH & RICHARDSON P.C., P.O BOX 1022, Minneapolis, MN, 55440-1022, US

(73) Assignee: THE UAB RESEARCH FOUNDATION, Birmingham, AL (US)

(21) Appl. No.: 12/514,088

(22) PCT Filed: Nov. 9, 2007
(86) PCT No.: PCT/US07/84240

§ 371 Date: May 8, 2009

Related U.S. Application Data
(60) Provisional application No. 60/865,265, filed on Nov. 10, 2006.
(60) Provisional application No. 60/941,856, filed on Jun. 4, 2007.

(51) Int. Cl. A61K 31/7088; C07H 21/02; C12N 5/10; A01K 67/00; C12Q 1/68; C12N 5/02; A61P 9/00

(52) U.S. Cl. 514/44 R; 536/23.1; 435/325; 800/13; 435/6; 435/375

(57) ABSTRACT

Provided herein is a microRNA, specifically, β-myosin microRNA, compositions comprising β-myosin microRNA and methods of inhibiting or reducing expression of β-myosin microRNA. Also provided are genetically modified cells and animals comprising exogenous β-myosin microRNA. Provided herein are methods of screening for agents that modulate β-myosin microRNA expression. In addition, methods of identifying target genes that are regulated by β-myosin microRNA and methods of modulating such genes are described. Methods of diagnosing or determining whether a subject is at risk for a cardiovascular disorder are also described.

### CROSS-REFERENCE TO RELATED APPLICATIONS

This application claims priority to U.S. Provisional Application No. 60/865,265 filed Nov. 10, 2006 and U.S. Provisional Application No. 60/941,856, filed Jun. 4, 2007, which are incorporated by reference herein in their entireties.

### STATEMENT REGARDING FEDERALLY SPONSORED RESEARCH

This invention was made with government support under Grant No. R01-HL66911 from the National Heart, Lung and Blood Institute. The government may have certain rights in the invention.

### TECHNICAL FIELD

This disclosure relates to microRNAs and methods, compositions and research tools related to microRNAs, and more particularly to β-myosin microRNA and methods, compositions and research tools related to β-myosin microRNA.

### BACKGROUND

MicroRNAs (referred to as miRNAs) are small non-coding RNAs, belonging to a class of regulatory molecules found in plants and animals. Generally, miRNAs control gene expression by binding to complementary sites on target messenger RNA (mRNA) transcripts. mRNAs are generated from large RNA precursors (termed pri-miRNAs), which are processed in the nucleus into approximately 70 nucleotide pre-miRNAs. Pre-miRNAs fold into imperfect stem-loop structures and undergo an additional processing step within the cytoplasm where mature miRNAs of 18-25 nucleotides in length are excised from one side of the pre-miRNA hairpin by an RNase III enzyme, Dicer. mRNAs have been shown to regulate gene expression in two ways. First, miRNAs that bind to protein-coding mRNA sequences that are exactly complementary to the miRNA induce the RNA-mediated interference (RNAi) pathway. Messenger RNA targets are cleaved by ribonucleases in the RISC complex. This mechanism of miRNA-mediated gene silencing has been observed in plants and animals. In the second mechanism, miRNAs that bind to imperfect complementary sites on messenger RNA transcripts direct gene regulation at the posttranscriptional level but do not cleave their mRNA targets. mRNAs identified in both plants and animals use this mechanism to exert translational control of their gene targets.

Hundreds of miRNAs have been identified in the fly, worm, plant and mammalian genomes. mRNAs have been shown to act as important gene regulators, for example, for tissue growth and development, cell differentiation, insulin secretion and regulation of viral infections. Therefore, miRNAs are important in a variety of developmental and metabolic processes.

### SUMMARY

Provided is a microRNA referred to herein as β-myosin microRNA and methods of modulating expression of β-myosin microRNA. Also provided herein are methods of screening for agents that modulate β-myosin microRNA expression. In addition, methods of identifying target genes that are regulated by β-myosin microRNA and methods of modulating such genes are described. Methods of diagnosing or determining whether a subject is at risk for a cardiovascular disorder are also described.

### DESCRIPTION OF DRAWINGS

FIG. 1 shows the structure and sequence of the mature β-myosin microRNA. The cardiac β-myosin heavy chain (HC) gene promoter is shown schematically at the top. The solid box shows the first exon of the β-myosin HC gene with the arrow showing the direction of transcription of that gene. The microRNA (shaded box) is located upstream of the first exon. It is derived from an RNA whose transcription is initiated in the promoter sequences at approximately −170 in humans (approximately-160 in rabbits) upstream from the capsite and proceeds in the antisense direction. The sequence of the 21 nucleotide mature microRNA is shown below (SEQ ID NO:1). Also shown are the sequences of the corresponding genomic regions from the human (SEQ ID NO:2), rabbit (SEQ ID NO:3) and mouse genomes (SEQ ID NO:4).

FIG. 2 is a gel showing divergent transcripts produced from the β-myosin heavy chain promoter in human hearts of diabetic, heart failure and normal subjects.

FIGS. 3A and 3B are graphs showing representative QT-PCR amplification profiles for the analyses of β-myosin microRNA and mir1-1 from a failing (F) and a nonfailing (NF) human heart indicated by the arrows.

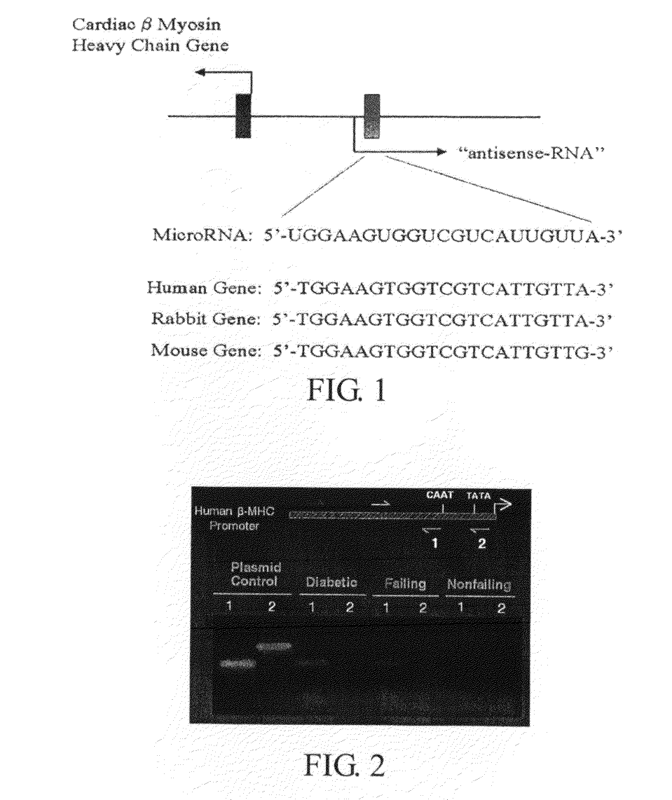

### DETAILED DESCRIPTION

Described herein is a microRNA transcribed antisense to the β-myosin heavy chain gene promoter, referred to herein as β-myosin microRNA and methods of using the β-myosin microRNA. β-myosin microRNA is induced in the failing heart and its expression appears to switch on cardiomyopathic alterations in the heart. β-myosin microRNA thus serves as a therapeutic target. In addition, target genes of the β-myosin microRNA have been identified, which are therapeutic targets for the treatment of cardiomyopathy and heart failure. As used herein, the term β-myosin microRNA gene refers to the nucleic acid encoding the β-myosin microRNA. Homologues and variants thereof include conservative substitutions, additions, and deletions therein not adversely affecting the structure or function. Preferably, the β-myosin microRNA gene refers to the nucleic acid encoding β-myosin microRNA. Biologically active sequence variants of β-myosin microRNA include alleles and in vitro generated derivatives of the β-myosin microRNA gene that encode β-myosin microRNA activity.

As used herein, the term microRNA refers to any type of interfering RNA, including but not limited to, endogenous microRNA and artificial microRNA. Endogenous microRNA are small RNAs naturally present in the genome which are capable of modulating the productive utilization of mRNA. The term artificial or non-naturally occurring microRNA includes any type of RNA sequence, other than endogenous microRNA, which is capable of modulating the productive utilization of mRNA.

Sequence variants of β-myosin microRNA fall into one or more of three classes: substitutional, insertional or deletional variants. Insertions include 5′ and/or 3′ terminal fusions as well as intrasequence insertions of single or multiple residues. Insertions can also be introduced within the mature sequence of β-myosin microRNA. These, however, ordinarily will be smaller insertions than those at the 5′ or 3′ terminus, on the order of 1 to 4 residues.

Insertional sequence variants of β-myosin microRNA are those in which one or more residues are introduced into a predetermined site in the target β-myosin microRNA. Deletion variants are characterized by the removal of one or more residues from the β-myosin microRNA sequence. These variants ordinarily are prepared by site specific mutagenesis of nucleotides in the DNA encoding β-myosin microRNA, thereby producing DNA encoding the variant, and thereafter expressing the DNA in recombinant cell culture. However, variant β-myosin microRNA fragments may be conveniently prepared by in vitro synthesis. The variants typically exhibit the same qualitative biological activity as the naturally-occurring analogue, although variants also are selected in order to modify the characteristics of β-myosin microRNA.

Substitutional variants are those in which at least one residue sequence has been removed and a different residue inserted in its place. While the site for introducing a sequence variation is predetermined, the mutation per se need not be predetermined. For example, in order to optimize the performance of a mutation at a given site, random mutagenesis may be conducted at the target region and the expressed β-myosin microRNA variants screened for the optimal combination of desired activity. Techniques for making substitution mutations at predetermined sites in DNA having a known sequence are well known.

Nucleotide substitutions are typically of single residues; insertions usually will be on the order of about 1 to 10 residues; and deletions will range about from 1 to 30 residues. Deletions or insertions preferably are made in adjacent pairs; i.e. a deletion of 2 residues or insertion of 2 residues. Substitutions, deletion, insertions or any combination thereof may be combined to arrive at a final construct.

Provided herein are also variants, modifications or derivatives of the disclosed β-myosin microRNA. It is understood that one way to define any variants, modifications, or derivatives of the disclosed nucleic acids herein is through defining the variants, modification, and derivatives in terms of identity to specific known sequences. As used herein, homologue refers to a nucleic acid with identity to a specific known sequence. Specifically disclosed are variants of the nucleic acids herein disclosed which have at least 40, 41, 42, 43, 44, 45, 46, 47, 48, 49, 49, 50, 51, 52, 53, 54, 55, 56, 57, 58, 59; 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent identity to the stated or known sequence. Those of skill in the art readily understand how to determine the identity of two nucleic acids. For example, the identity can be calculated after aligning the two sequences so that the identity is at its highest level.

A way of calculating identity can be performed by published algorithms. Optimal alignment of sequences for comparison is conducted, for example, by the local identity algorithm disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

As used herein the term nucleic acid refers to multiple nucleotides (i.e. molecules comprising a sugar (e.g. ribose or deoxyribose) linked to a phosphate group and to an exchangeable organic base, which is either a substituted pyrimidine (e.g. cytosine (C), thymidine (T) or uracil (U)) or a substituted purine (e.g. adenine (A) or guanine (G)). The term also includes polynucleosides (i.e. a polynucleotide minus the phosphate) and any other organic base containing polymer. Purines and pyrimidines include but are not limited to adenine, cytosine, guanine, thymidine, inosine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. Other such modifications are well known to those of skill in the art. Thus, the term nucleic acid also encompasses nucleic acids with substitutions or modifications, such as in the bases and/or sugars.

The nucleic acids disclosed herein optionally comprise nucleic acid analogs and/or modified internucleoside linkages. Nucleic acids containing modified internucleoside linkages may be synthesized using reagents and methods that are well known in the art. For example, methods of synthesizing nucleic acids containing phosphonate phosphorothioate, phosphorodithioate, phosphoramidate methoxyethyl phosphoramidate, formacetal, thioformacetal, diisopropylsilyl, acetamidate, carbamate, dimethylene-sulfide dimethylene-sulfoxide (—CH₂—SO—CH₂), dimethylene-sulfone (—CH₂—SO₂—CH₂), 2′-O-alkyl, and 2′-deoxy-2′-fluoro phosphorothioate internucleoside linkages are well known in the art.

Described herein is a microRNA referred to as β-myosin microRNA that mediates cardiomyopathic alterations. The gene is located within the promoter of the cardiac β-myosin heavy chain (HC) gene. β-myosin microRNA is highly conserved in a number of mammalian species. β-myosin microRNA is involved in altering the structure and function of cardiac tissue. The expression of the micro-RNA is selectively induced in human failing hearts and also in hearts of subjects with diabetes (see Example 1 and FIG. 2). The introduction of the microRNA gene alone into transgenic mice and the expression of the exogenous gene result in cardiomyopathic alterations associated with heart failure. Finally, in a transgenic mouse line, where the expression of the exogenous microRNA is turned on or turned off by altering the parental lineage of the transgene, only animals expressing the microRNA exhibit the cardiomyopathic alterations. Therefore, in both human clinical specimens and an experimental transgenic mouse model, the expression of β-myosin microRNA gene is responsible for the structural and functional changes intrinsic to cardiac failure and cardiomyopathy.

MicroRNAs modulate cell function, development, and fate by interacting with sequences of specific mRNAs and inhibiting the expression of the targeted mRNAs. Using published algorithms, databases of 3′-untranslated mRNA sequences were screened and a set of genes were identified whose expression is modulated by β-myosin micro-RNA. The potential target genes encode transcription factors, signal transduction proteins, splicing factors, ion channels and pumps, and cell surface proteins. Some of these genes have been implicated in mediating cardiomyopathic alterations in the failing heart.

βmyosin microRNA gene is encoded in the promoter region of the cardiac β-myosin HC gene. Transcription of the β-myosin microRNA gene starts at approximately 160 nucleotides (nts) in rabbits and 170 nucleotides in humans upstream of the start site of the cardiac β-myosin HC gene and proceeds in the antisense orientation. Antisense RNAs have been reported from various regions of the cardiac β- and α-myosin HC genes. Although the promoter sequences in humans and rabbits are slightly different, the sequences of the mature 21-nucleotide microRNAs in both species are identical.

The promoter of the cardiac β-myosin HC gene encodes a function that mediates cardiomyopathic alterations. Introduction of the promoter into transgenic mice results in a number of alterations in heart structure and function associated with heart failure. These include changes in heart morphology, diminished systolic and contractile function, an increase in mitochondria, impaired oxidative phosphorylation, and changes in intracellular calcium homeostasis in the heart. Along with diminished heart function, the transgenic animals also exhibit a number of systemic changes associated with cardiac failure. The latter include increased fluid flow in the lungs, altered vasomotor reactivity, autonomic dysfunction, glucose intolerance and hyperinsulinemia, and poor exercise tolerance. All of the phenotypic changes are due to the promoter sequences themselves, as transgenic mice containing transgenes in which the promoter sequences have been removed are normal. Furthermore, the cardiomyopathy exhibits an autosomal dominant mode of inheritance, is reproduced in independent transgenic mouse lines, and in a preliminary deletion analysis in transgenic mice, is mediated by a region from −150 to −300 of the rabbit cardiac β-myosin HC gene promoter. These results indicate that cardiac β-myosin HC gene promoter mediates many of the cardiomyopathic alterations observed in heart failure and cardiomyopathy.

Expression of β-myosin microRNA is inducible and correlated with the modification of the promoter by DNA methylation. In transgenic mice where methylation of the cardiac β-myosin HC promoter transgene is altered, only animals with a methylated transgene exhibit the cardiomyopathic alterations whereas animals with a hypomethylated transgene have a normal heart phenotype. Thus, DNA methylation appears to be the switch that allows β-myosin microRNA to be expressed. The dependence of the cardiomyopathic function on DNA methylation may explain why β-myosin microRNA is not normally expressed.

Alterations in DNA methylation of the endogenous β-myosin HC gene promoter also occur in clinical heart failure. Analyses of DNA from human failing and non-failing hearts indicate that site-specific increases in DNA methylation of endogenous human cardiac β-myosin HC gene promoter are correlated with clinical heart failure. These changes in DNA methylation do not appear to be a consequence of the pathology as similar changes in DNA methylation are also observed in diabetic cardiomyopathy (in the absence of overt heart failure), and, in an experimental animal model, may be initiated during the cardiac growth adaptation preceding cardiac failure. Thus, β-myosin microRNA expression appears to be a primary event initiating alterations of the structure and function of the heart. The ability of the transgenic mouse model to predict a previously unknown epigenetic alteration in human cardiomyopathic and failing hearts also indicates the clinical relevance of processes occurring in this model.

Transcription of the β-myosin microRNA is initiated in the transgene promoter sequence, its expression is associated with methylation of the promoter sequence, and the expression is correlated with cardiomyopathic alterations. In human hearts, the expression of β-myosin microRNA is expressed from endogenous cardiac β-myosin HC gene that is induced in failing hearts, and also in hearts of diabetic subjects. The experimental data and clinical analyses indicate that fβ-myosin microRNA mediates cardiomyopathic alterations in the heart.

Using an algorithm developed to identify potential microRNA-target sequence interactions (MiRanda), targets for the fβ-myosin microRNA were identified. (3-myosin microRNA target genes include immunglobin transcription factor (ITF)4, ITF2, adapter complex mu3A subunit, HIV-1 rev-binding protein-like protein, SOX-1 3 protein, ras-related protein M-Ras, protocadherin, serinelarginine-rich protein kinase 2, calcium transporting ATPase, type 2C, dual specificity protein phosphatase 6, serine/threonine protein phosphatase 2A, catalytic subunit, sarcoplasmic reticulum calcium ATPase-2, glucoronosyl-N-acetylglucosaminyl-proteoglycan 4-alpha-N-acetylglucosaminyltransferase, misshapenINIK-related kinase-2, neuroendocrine secretory protein 55, homeobox protein HOX-B5, calpactin heavy chain, fantom protein, melanoma antigen, transcription factor Sp3, splicing factor SC35, potassium channel modulatory protein, RNA binding motif protein 5, neuritin, G-protein beta1 subunit, ubiquitin-conjugating enzyme E2 (Ube2W), p21 activated Kinase-3 (MPAK3), organic anion transporter MOAT 1, TREK-1 2 pore domain potassium channel and ras-related protein Rab21. The list contains a number of genes whose expression is altered in the failing heart. These include transcription factors, RNA splicing factors and protein kinases, signal transduction proteins, and calcium and other ion transporters. In some instances, the inactivation of the respective endogenous gene in mice by homologous recombination results in a dilated cardiomyopathy and heart failure.

Numerous studies point to alterations in gene expression leading to changes in calcium cycling in the cell, ion channel movements, oxidative phosphorylation, switches in alternative splicing and signal transduction pathways in the failing heart. While all are important in altering the structure and function of the failing heart, it is not clear which if any is primary event in leading to changes in the heart. Described herein is a microRNA that regulates the expression of a number of genes that have been shown to have an impact on the structure and function of the failing heart. β-myosin microRNA can be used in the methods described herein to identify other target genes that mediate cardiomyopathic alterations in the heart.

Provided herein are methods for reducing or inhibiting expression of β-myosin microRNA. For example, β-myosin microRNA is inhibited by antisense molecules, which are designed to interact with a target molecule, in this case the β-myosin microRNA or the β-myosin microRNA gene, through either canonical or non-canonical base pairing. The interaction of the antisense molecule and the β-myosin microRNA is designed to promote the destruction of the target molecule through, for example, RNAseH mediated RNA-DNA hybrid degradation. Alternatively the antisense molecule is designed to interrupt a processing function that normally would take place on the β-myosin microRNA gene, such as transcription or replication. Antisense molecules are designed based on the sequence of the target molecule. Numerous methods for optimization of antisense efficiency by finding the most accessible regions of the target molecule exist. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules is found in the art.

Triplex forming functional nucleic acid molecules are molecules that interact with either double-stranded or single-stranded nucleic acid to introduce mutations that inactivate a gene or inhibit transcription. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹°, or 10⁻¹². Representative examples of how to make and use triplex forming molecules to bind a variety of different target molecules is found in the art.

Short Interfering RNA (siRNA) is a double-stranded RNA that induces sequence-specific post-transcriptional gene silencing, thereby decreasing or even inhibiting gene expression. In one example, an siRNA triggers the specific degradation of homologous RNA molecules, such as mRNAs, within the region of sequence identity between both the siRNA and the target RNA. For example, WO 02/44321 discloses siRNAs capable of sequence-specific degradation of target mRNAs when base-paired with 3′ overhanging ends, herein incorporated by reference for the method of making these siRNAs. Sequence specific gene silencing is achieved in mammalian cells using synthetic, short double-stranded RNAs that mimic the siRNAs produced by the enzyme dicer. siRNA can be chemically or in vitro-synthesized or can be the result of short double-stranded hairpin-like RNAs (shRNAs) that are processed into siRNAs inside the cell. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech (Esbersberg, Germany), Proligo (Boulder, Colo.), and Qiagen (Vento, Netherlands). siRNA can also be synthesized in vitro using kits such as Ambion's SILENCER® siRNA Construction Kit (Ambion, Austin, Tex.).

The production of siRNA from a vector is more commonly done through the transcription of a short hairpin RNAs (shRNAs). Kits for the production of vectors comprising shRNA are available, such as, for example, Imgenex's GENESUPPRESSOR™ Construction Kits (Imgenex Corporation, San Diego, Calif.) and Invitrogen's BLOCK-ITT™ inducible RNAi plasmid and lentivirus vectors (Invitrogen, Carlsbad, Calif.).

Antisense oligonucleotides (ASO), also known as antagomirs when applied to inhibiting miRNAs, are used for inhibiting miRNAs. Antigomers are single-stranded ribonucleotides of 21-23 nucleotides in length, which are complementary to the target miRNA. Antagomirs are often 2′-O-methyl-modified to enhance inhibition of miRNAs. In addition, conjugating cholesterol to the antigomir results in increased efficiency in miRNA silencing (Krutzfeldt et al., Nature 438:685-689 (2005)). Cholesterol-conjugated antagomirs have been shown to specifically inhibit miRNAs in the heart (Care et al., Nature Medicine 13(5):613-618 (2007)). Thus, provided herein are antagomirs that target β-myosin miRNA. The antagomirs are optionally targeted to cholesterol to inhibit β-myosin miRNA.

Described herein are cell- and animal-based models for cardiovascular disease. These models are useful, for example, to further characterize the β-myosin microRNA. These models are useful in screening assays to identify agents which are capable of ameliorating cardiovascular disease symptoms. Thus, the animal- and cell-based models are used to identify drugs, pharmaceuticals, therapies and interventions which are effective in treating cardiovascular diseases, and to determine the toxicity and bioavailability where such data are used to determine the in vivo efficacy of potential cardiovascular disease treatments. The term transgenic animal refers to an animal that contains within its genome a specific gene that has been disrupted or altered, in this case, β-myosin microRNA. The transgenic animal includes both the heterozygote animal (i.e., one defective allele and one wild-type allele) and the homozygous animal (i.e., two defective alleles).

Animal-based models of cardiovascular disease are provided that include, but are not limited to, non-recombinant and engineered transgenic animals. The animal models exhibiting cardiovascular disease symptoms, for example, are engineered to overexpress β-myosin microRNA using techniques for producing transgenic animals that are well known to those of skill in the art. The β-myosin microRNA gene sequence can be introduced into, overexpressed in, repressed in, or knocked out in the genome of the cell or animal of interest.

Animals of any species, including, but not limited to, mice, rats, rabbits, guinea pigs, pigs, micro-pigs, goats, and non-human primates, such as baboons, monkeys, and chimpanzees are useful for generating cardiovascular disease animal models.

Any technique that disrupts, alters or overexpresses the β-myosin microRNA gene in cells and transgenic animals is useful in the methods provided herein. Such techniques include, but are not limited to pronuclear microinjection (U.S. Pat. No. 4,873,191); retrovirus mediated gene transfer into germ lines (Van der Putten et al., Proc. Natl. Acad. Sci., USA 82:6148-6152 (1985)); gene targeting in embryonic stem cells (Thompson et al., Cell 56:313-321 (1989)); electroporation of embryos (Lo, Mol Cell. Biol. 3:1803-1814 (1983)); and other techniques used in the art and reviewed in Gordon, Intl. Rev. Cytol. 115:171-229 (1989).

To disrupt β-myosin microRNA, targeting constructs are used that typically contain selectable markers surrounded by two regions of identity to the targeted locus. Homologous recombination between the genome and the two regions of identity result in the replacement of the targeted locus with the targeting construct. PCR and/or Southern blotting are employed to identify those cells and animals that have correctly incorporated the targeting construct into the genome.

β-myosin microRNA function is reduced by administering a composition, such as an isolated oligonucleotide or small molecule that interferes with β-myosin microRNA activity. An oligonucleotide that interferes with β-myosin microRNA activity include, for instance, an oligonucleotide that is substantially antisense to an β-myosin microRNA and/or a portion thereof such as, for example, an antagomir. Other types of oligonucleotides that interfere with the β-myosin microRNA activity include oligonucleotides that are antisense to the miRNA binding region of the β-myosin microRNA.

Once transgenic animals have been generated, the expression of the β-myosin microRNA is assayed utilizing standard techniques. Initial screening, for example, is accomplished by Southern blot analysis or PCR techniques to analyze animal tissues to assay whether integration of the transgene has taken place.

The level of β-myosin microRNA expression is assessed using techniques which include, but are not limited to, Northern blot analysis of tissue samples obtained from the animals, in situ hybridization analysis, QT-PCR, stem-loop RT-PCR and RT-PCR.

Cells that contain the β-myosin microRNA and exhibit cellular phenotypes associated with cardiovascular disease, are utilized to identify agents that exhibit anti-cardiovascular disease activity. Such cells include cardiomyocytes and cardiomyocyte precursor cells. Further, such cells include recombinant, transgenic cell lines. For example, the cardiovascular animal models of the invention described in detail above, are used to generate cell lines, containing one or more cell types involved in cardiovascular disease, that can be used as cell culture models for this disorder.

Cells of a cell type known to be involved in cardiovascular disease are optionally transfected with sequences or contacted with agents capable of decreasing β-myosin microRNA expression within the cell. For example, cardiac myocytes to be transfected include primary cultures of neonatal cardiomyocytes or primary cultures of adult cardiac myocytes, AT-1 cardiomyocytes (atrial tumor cells), and mouse P19 embryonal carcinoma cells. Cardiac myocytes to be transfected also include derivatives of AT-1 tumor cells such as, for example, HL-1 cells.

Screening assays for agents that interact with the β-myosin microRNA and/or that modulate its expression are provided. Assays to identify agents that bind to the β-myosin microRNA or bind to other cellular or extracellular mRNA that interact with the β-myosin microRNA are provided. Agents identified using such assays ameliorate cardiovascular diseases, such as, for example, heart conditions, atherosclerosis, ischemia/reperfusion, hypertension, restenosis, and arterial inflammation by modulating the activity of β-myosin microRNA. Such agents include, but are not limited to nucleic acids, peptides, antibodies, small organic agents, or inorganic agents. Agents identified are useful, for example, in modulating the activity of βmyosin microRNA.

Methods for screening candidate agents for their ability to antagonize the interaction between β-myosin microRNA and its target genes are provided. The method involves a) contacting a candidate agent with a cell or animal expressing β-myosin microRNA and a target gene of interest and b) determining whether the compound interferes with the interaction between β-myosin microRNA and the target gene. The method optionally comprises evaluating expression levels of RNA or protein encoded by the target gene after the candidate agent has been contacted with a cell or animal expressing β-myosin microRNA. The determining step is accomplished by measuring one or more phenotypes associated with cardiovascular disorders.

The agents that inhibit β-myosin microRNA identified by the methods described above are tested for the ability to ameliorate cardiovascular disease symptoms using, for example, cell-based and animal model-based assays. Phenotypic changes associated with cardiovascular diseases or cardiovascular disease symptoms include, but are not limited to, dilated heart, moderate hypertrophy, increased distensibility, decreased collagen, abnormal EKG, increased end-diastolic dimension, depressed systolic function, decreased left ventricle fractional shortening, altered histology, increased mitochondria, myofibrillar disarray, impaired oxidative phosphorylation, depressed contractility, changes in myosin-ATPase, myofilament calcium sensitivity, altered gene expression, decreased SR Ca-ATPase mRNA, distension of peribreonchial and perivascular lymphatics, altered coronary vascular responses, metabolic alterations including impaired glucose tolerance, increased serum triglycerides and islet hyperplasia and reduced exercise tolerance.

Cell-based models are used to identify agents that ameliorate cardiovascular disease symptoms. For example, such cell-based models are exposed to a compound followed by measuring expression of β-myosin microRNA in the exposed cells. Agents that inhibit or reduce expression of β-myosin microRNA in the exposed cells are useful for ameliorating cardiovascular disease symptoms. The cells are examined to determine whether one or more of the cardiovascular disease cellular phenotypes has been altered to resemble a more normal or more wild type, non-cardiovascular disease phenotype.

Transgenic animals are also used to identify agents capable of ameliorating cardiovascular disease symptoms. The animal models can be used to identify drugs, pharmaceuticals, therapies, and interventions effective in treating cardiovascular disease. For example, animal models are exposed to a compound followed by measuring expression of β-myosin microRNA in the exposed animals. Agents that inhibit or reduce expression of β-myosin microRNA in the exposed animals are useful for ameliorating cardiovascular disease symptoms. The response of the animals to the exposure is monitored by assessing the reversal of disorders associated with cardiovascular disease, for example, by counting the number of atherosclerotic plaques and/or measuring their size before and after treatment.

The effects of the agents on cardiovascular disease states, such as in clinical trials, is monitored. Thus, in a clinical trial where the patients are administered the test drug, levels of expression of β-myosin microRNA is quantified, for example, by Northern blot analysis or RT-PCR. Thus, these profiles serve as surrogate markers indicative of the physiological response, and are determined before, and at various points during, drug treatment.

In general, agents that inhibit β-myosin microRNA are optionally identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries and libraries of natural compounds in the form of bacterial, fungal, plant, and animal extracts are commercially available. In addition, natural and synthetically produced libraries are generated, if desired, according to methods known in the art, e.g., by standard extraction and fractionation methods. Furthermore, if desired, any library or compound is readily modified using standard chemical, physical, or biochemical methods.

Methods of predicting the relative risk of a subject developing a cardiovascular disease or disorder, such as, for example, heart failure or cardiomyopathy, comprise obtaining a sample from the subject and determining the level of expression of β-myosin microRNA, wherein an increase in β-myosin microRNA as compared to control indicates the subject is a risk for developing heart failure or cardiomyopathy. As used herein the term sample includes any biological sample, such as, for example, tissue or biological fluid. Methods of diagnosing a cardiovascular disease or disorder in a subject, comprise obtaining a sample from the subject and determining the level of expression of β-myosin microRNA, wherein an increase in β-myosin microRNA as compared to control indicates the subject is at risk for developing heart failure. The level of β-myosin microRNA expression is assessed using techniques which include but are not limited to Northern blot analysis of tissue samples obtained from the animals, in situ hybridization analysis, QT-PCR, stem-loop RT-PCR and RT-PCR.

The provided methods optionally comprise the administration and uptake of exogenous DNA into the cells of a subject (i.e., gene transduction or transfection). The disclosed nucleic acids are in the form of, for example, naked DNA or RNA, or the nucleic acids are in a vector under the transcriptional regulation of a promoter for delivering the nucleic acids to the cells. The vector is optionally a commercially available preparation, such as an adenovirus vector. Delivery of the nucleic acid or vector to cells is via a variety of mechanisms. As one example, delivery is via a liposome, using commercially available liposome preparations, as well as other liposomes developed according to procedures standard in the art. As another example, the disclosed nucleic acid or vector is delivered in vivo by electroporation.

As one example, vector delivery is via a viral system, such as a retroviral vector system that can package a recombinant retroviral genome. The recombinant retrovirus is used to infect and thereby deliver to the infected cells the nucleic acid. The exact method of introducing the altered nucleic acid into mammalian cells is, of course, not limited to the use of retroviral vectors. Other techniques are widely available for this procedure including the use of adenoviral vectors, lentiviral vectors, pseudotyped retroviral vectors. Physical transduction techniques are optionally used, such as liposome delivery and receptor-mediated and other endocytosis mechanisms. This disclosed compositions and methods are optionally used in conjunction with any of these or other commonly used gene transfer methods.

As one example, if the nucleic acid is delivered to the cells of a subject in an adenovirus vector, the dosage for administration of adenovirus to humans ranges from about 10³ to 10¹² or any amount between 10³ to 10¹² or from about 10⁷ to 10⁹ plaque forming units (pfu) per injection, but can be as high as 10¹⁵ pfu per injection. A subject receives a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector, if used, is generally characterized by injection. Injectables are generally prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. For additional discussion of suitable formulations and various routes of administration of therapeutic compounds, see, e.g., Remington: The Science and Practice of Pharmacy (21st ed.) eds. A. R. Gennaro et al., University of the Sciences in Philadelphia 2005.

Effective dosages and schedules for administering the compositions can be determined empirically, and making such determinations is within the skill in the art. The dosage ranges for the administration of the compositions are those large enough to produce the desired effect in which the symptom's disorder are affected. The dosage should not be so large as to cause adverse side effects, such as unwanted cross-reactions, anaphylactic reactions, and the like. Generally, the dosage varies with the age, condition, sex and extent of the disease in the patient, route of administration, or whether other drugs are included in the regimen, and are determined by one of skill in the art. The dosage is adjusted by the individual physician in the event of any counterindications. Dosage varies and are administered, for example, in one or more dose administrations daily, for one or several days. Guidance is found in the literature for appropriate dosages for given classes of pharmaceutical products. A typical daily dosage of the provided compositions used alone range, for example, from about 1 μg/kg to up to 100 mg/kg of body weight or more per day, depending on the factors mentioned above.

Following administration of a disclosed composition for treating, inhibiting, or preventing a cardiovascular disease or disorder, the efficacy of the therapeutic composition is assessed in various ways. For instance, one of ordinary skill in the art determines if a composition is efficacious in treating or inhibiting cardiovascular diseases in a subject using an electrocardiogram (ECG/EKG), echocardiogram (ECHO), or MRI.

The compositions disclosed herein to perform the disclosed methods are made using a variety of methods for that particular reagent or compound. For example, the nucleic acids are made using standard chemical synthesis methods or produced using enzymatic methods or other method. Such methods include standard enzymatic digestion followed by nucleotide fragment isolation (see, for example, Sambrook et al., Molecular Cloning: A Laboratory Manual, 2nd Edition (Cold Spring Harbor Laboratory Press, Cold Spring Harbor, N.Y., 1989) Chapters 5, 6) or purely synthetic methods, for example, by the cyanoethyl phosphoramidite method. Synthetic methods useful for making oligonucleotides also include, but are not limited to, phosphotriester and phosphite-triester methods and Narang phosphotriester method.

The β-myosin microRNA gene or β-myosin microRNA described above is optionally in a formulation for administration topically, locally or systemically in a suitable pharmaceutical carrier. The nucleic acids are optionally encapsulated in suitable biocompatible microcapsules, microparticles or microspheres formed of biodegradable or non-biodegradable polymers or proteins or liposomes for targeting to cells. Such systems are optionally optimized for use with the appropriate nucleic acid.

Nucleic acids administered in vivo are taken up and distributed to cells and tissues (Huang, et al., FEBS Lett. 558(1-3):69-73 (2004)). For example, Nyce et al. have shown that antisense oligodeoxynucleotides (ODNs) when inhaled bind to endogenous surfactant (a lipid produced by lung cells) and are taken up by lung cells without a need for additional carrier lipids (Nyce and Metzger, Nature, 385:721-725 (1997). Small nucleic acids are readily taken up into T24 bladder carcinoma tissue culture cells (Ma, et al., Antisense Nucleic Acid Drug Dev. 8:415-426 (1998). siRNAs have been used for therapeutic silencing of an endogenous genes by systemic administration (Soutschek, et al., Nature 432, 173-178 (2004)).

As used herein, subject includes, but is not limited to, a vertebrate, more specifically a mammal (e.g., a human, horse, pig, rabbit, dog, sheep, goat, non-human primate, cow, cat, guinea pig or rodent), a fish, a bird or a reptile or an amphibian. The term does not denote a particular age or sex. Thus, adult and newborn subjects, as well as fetuses, whether male or female, are intended to be covered. The term subject includes human and veterinary subjects.

Unless defined otherwise, all technical and scientific terms used herein have the same meanings as commonly understood by one of skill in the art to which the disclosed method and compositions belong. Publications cited herein and the material for which they are cited are hereby specifically incorporated by reference. No admission is made that any reference constitutes prior art. The discussion of references states what their authors assert, and applicants reserve the right to challenge the accuracy and pertinence of the cited documents. It will be clearly understood that, although a number of publications are referred to herein, such reference does not constitute an admission that any of these documents forms part of the common general knowledge in the art.

Inhibit, inhibiting and inhibition mean to decrease expression, an activity, response, condition, disease, or other biological parameter. This includes, but is not limited to, the complete ablation of the expression, activity, response, condition, or disease. This may also include, for example, a 10% reduction in expression, activity, response, condition, or disease as compared to the native or control level. Thus, the reduction can be a 10, 20, 30, 40, 50, 60, 70, 80, 90, 100%, or any amount of reduction in between as compared to native or control levels.

Throughout the description and claims of this specification, the word comprise and variations of the word, such as comprising and comprises, means including but not limited to, and is not intended to exclude, for example, other additives, components, integers or steps.

A number of aspects have been described. Nevertheless, it will be understood that various modifications may be made. Furthermore, when one characteristic or step is described it can be combined with any other characteristic or step herein even if the combination is not explicitly stated. Accordingly, other aspects are within the scope of the claims.

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure.

### EXAMPLES

### Example 1

### A MicroRNA Encoded by the Beta-Myosin Heavy Chain Promoter is Associated with Cardiomyopathic Alterations.

Transgenic mice containing sequences of the rabbit β-myosin heavy chain gene promoter exhibit many of the structural and functional alterations of heart failure. These phenotypic changes such as diminished contractile function, altered calcium homeostasis and an excess of mitochondria are mediated by an antisense RNA transcribed from the promoter sequences. To elucidate the mechanism by which this antisense RNA mediates its effect, the structure of the antisense RNA was characterized by amplifying by PCR and cloning of its terminal sequences. The analyses indicated transcription of the antisense RNA occurs at approximately 160 base pairs upstream of the capsite of the β-myosin heavy chain gene and extends throughout the length of the transgene promoter sequences. In some transgenic lines, the antisense RNA terminates following a cryptic polyadenylation signal in the adjoining gene sequences. The analyses also identified a small RNA of 21 nucleotides (nt) encoded by the rabbit promoter and derived from the antisense RNA transcript. Sequences of this 21-nt RNA form a hairpin structure with adjacent sequences and correspond to a microRNA. Using the algorithm, MIRANDA, the sequence of the 21-nt RNA was used to identify target mRNAs. Selecting only target sequences highly conserved in human, mouse, and rat mRNAs, approximately 20 target genes were identified. The list included genes such as transcription factors, splicing factors, signal transduction proteins and ion transporters. A number of these genes have been implicated in cardiomyopathy and heart failure. The genes included, but are not limited to, immunglobin transcription factor (ITF)4, ITF2, adapter complex mu3A subunit, HIV-1 rev-binding protein-like protein, SOX-1 3 protein, ras-related protein M-Ras, protocadherin, serinelarginine-rich protein kinase 2, calcium transporting ATPase, type 2C, cual specificity protein phosphatase 6, serine/threonine protein phosphatase 2A, catalytic subunit, sarcoplasmic reticulum calcium ATPase-2, glucoronosyl-N-acetylglucosaminyl-proteoglycan 4-alpha-N-acetylglucosaminyltransferase, misshapenINIK-related kinase-2, neuroendocrine secretory protein 55, homeobox protein HOX-B5, calpactin heavy chain, fantom protein, melanoma antigen, transcription factor Sp3, splicing factor SC35, potassium channel modulatory protein, RNA binding motif protein 5, neuritin, G-protein beta1 subunit, ubiquitin-conjugating enzyme E2 (Ube2W), p21 activated Kinase-3 (MPAK3), organic anion transporter MOAT 1, TREK-1 2 pore domain potassium channel, Pumilio homolog-2, Actin-related protein ⅔ complex subunit-5 like protein, Nedd4-like ubiquitin-protein ligase 1, Voltage-dependent L-type calcium channel beta-4 subunit, Transcription factor 12, Coronin-1C, Puromycin-sensitive aminopeptidase, Calcium/calmodulin-dependent protein kinase II alpha, Voltage-gated sodium channel type 1 beta subunit, LIM homeodomain protein cofactor (CLIM 1B), Guanine nucleotide binding protein Gi-alpha-2 subunit, Voltage-gated potassium channel subunit Kv1.3, Mitochondrial leucyl-tRNA synthetase, Myelin basic protein, splicing factor arginine/serine-rich 7, Exostosin-like 1, Sodium/glucose cotransporter, splicing factor arginine/serine-rich-4, Stanniocalcin-1 precursor, transcription factor SOX-17, human Erbb4, cAMP-dependent transcription factor ATF-7, PPAR gamma coactivator 1beta-1A, Neuronal PAS domain-containing protein, Splicing factor 1 (ZFP 162), TRAAK 2-pore domain channel, Neurogenin-1, CBP/P300-interacting transactivator, ADP-ribosylation factor 5, Nuclear receptor coactivator 1, Sodium/potassium transporting ATPase beta-1 subunit, Myelin Po protein precursor, Ataxin-10, ATP-dependent RNA helicase DDX5, Runt-related transcription factor 1, Serine/threonine-protein kinase PLK3, Cyclin L1, Mitochondrial citrate transporter, Homeobox protein D10(HOX-4D), Image clone 3448011 (probable DNA topoisomerase I) and ras-related protein Rab21. The results showed that a microRNA gene encoded by the β-myosin heavy chain promoter plays an important role in mediating cardiomyopathic alterations in the failing heart.

### Example 2

### Quantitative Real-Time Analysis of the β-Myosin MicroRNA

To quantify the β-myosin microRNA in a real-time PCR assay, an adapter oligonucleotide was ligated to the 3′-termini of RNAs. Complementary DNAs for the adapter-RNA chimeras were then generated with a reverse transcription primer (RT primer) that was complementary to a portion of the adapter oligonucleotide. The β-myosin microRNA was then quantified from the resultant cDNAs in a real-time PCR assay using a “nested” adapter primer (AP) and a gene-specific primer for the β-myosin microRNA (GP). The adapter primer and the gene-specific primers did not abut one another. Thus, the amplification of the adapter-RNA chimeras was distinguished from primer-dimer artifacts by the presence of junctional sequences unique to the ligation product. In the case of the β-myosin microRNA, ligation of the adapter oligonucleotide to the 3′-terminus also generated a unique site for the restriction enzyme, Stul. The size of the PCR fragment and the presence of a StuI site were used to verify the amplification of β-myosin microRNA in the real-time assay.

Since the adapter can be joined to the 3′-termini of other RNAs, and adapter containing cDNAs for those RNAs are generated by reverse transcription reaction, variations in the efficiency of ligation and reverse transcription were controlled by assessing the presence of another control RNA with gene-specific primers for that RNA. Examples of control RNA include, for example, 5S rRNA, u6 RNA, mir1-1 and mir16. In this example, microRNA, mir1-1 was chosen. Mir1-1 is one of the more abundant microRNAs expressed in the heart. Normalization of the amount of the β-myosin microRNA to the amount of mir1-1 provided an index of the relative change in expression of the β-myosin microRNA that is controlled for variations in sample preparation, ligation of the adapter oligonucleotide, and reverse transcription.

Specifically, RNA was isolated from ventricular tissue using the guanidium-isothiocyanate method, treated with RQ1 DNase (Promega, Madison, Wis.) according the manufacturer's protocol, extracted with phenol-chloroform, and precipitated with ethanol. The RNA (1-2 micrograms) was ligated to the adapter oligonucleotide with a blocked 3′-terminus (5′-pGGCCTCCCTTGGCTCGTTGGAG-3′-C6-amino linker (SEQ ID NO:5)) in a 20-4 reaction containing 1×T4 RNA ligase reaction buffer (New England BioLabs, Ipswich, Mass.), 2.6 μM adaptor oligonucleotide, 50% PEG-1000 (Sigma-Aldrich, St. Louis, Mo.), 26 units of RNasin (Promega) and 2 units of T4 RNA ligase (New England BioLabs; Ipswich, Mass.). The ligation mixture was incubated at 37° C. for 3 hours, then diluted to 100-4 with a solution of 0.05 mM aurin tricarboxylic acid, extracted twice with phenol-chloroform (1:1), and the RNA precipitated with ethanol using linear-acrylamide as a carrier. The precipitate was collected by centrifugation, dried, and dissolved in RNase-free water.

Complementary DNAs for the adapter-ligated RNAs were synthesized with Omniscript (Qiagen, Valencia, Calif.). A 20-KL reaction was assembled following the manufacturer's protocol along with 6 μM of the RT primer (5′-TACTCCAACGAGCCA-3′ (SEQ ID NO:6)) and 1 μg of the adapter-ligated RNA. Reverse transcription was at 37° C. for 60 minutes. The reaction mixture then was heated at 95° C. for 15 minutes. Prior to the real-time PCR analysis, the cDNA reactions were diluted 10-20 fold with a solution of 1 mM Tris HCl, pH 7.4, 0.5 mM EDTA.

Real-time PCR was performed on a DNA Engine Opticon 2 system (MJ Research/BioRad Laboratories, Hercules, Calif.). For the β-myosin microRNA, a 10-μL SYBR® green reaction contained 5 μL, of DYNAMO™ HS enzyme mixture (New England Biolabs), 140 nM adapter primer (5′-CGAGCCAAGGGAGGC-3′ (SEQ ID NO:7)), 106 nM β-myosin microRNA primer (5′-GGAAGTGGTCGTCATTGTTA-3′(SEQ ID NO:8)) and 1 μL diluted reverse transcription reaction. The reactions were heated at 95° C. for 12 minutes, followed by 50 cycles of 94° C. for 30 seconds and 60° C. for 35 seconds. Real-time PCR for mir1-1 was carried out under different conditions. A 10.6-μL contained 5 μL of DYNAMO™ HS enzyme mixture, 190 nM adapter primer 2 (5′-CTCAACGAGCCAAGG-3′ (SEQ ID NO:9)), 125 nM mir1-1 primer (5′-AATGGAATGTAAAGAAGTATGTA-3′ (SEQ ID NO:10)) and 1 μL diluted reverse transcription reaction. The mir1-1 reactions were heated at 95° C. for 12 minutes, followed by 55 cycles of 94° C. for 15 seconds, 46° C. for 20 seconds and 60° C. for 2 seconds. Quantification of the target RNAs was based on the amplification efficiency (Ramakers C, Ruijter J M, Lekanne Deprez R H, Moorman A F M. 2003. Assumption-free analysis of quantitative real-time polymerase chain reaction (PCR) data. Neurosci Lett 339, 62-66).

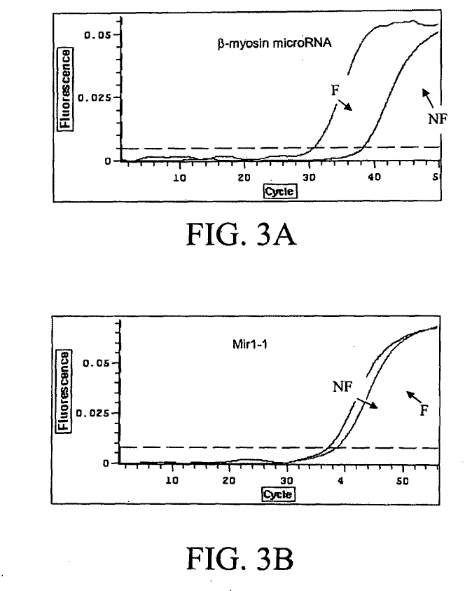

Representative amplification profiles for the analyses of RNAs from a failing (F) and a nonfailing (NF) human heart are shown in FIGS. 3A and 3B.

The expression of the β-myosin microRNA was also examined in human heart specimens. The clinical specimens were obtained at autopsy. Table 1 below shows the relative expression of the β-myosin microRNA in these tissues before and after normalization to mir1-1 levels in the same sample. The analyses were carried out in triplicate, and repeated at least twice. The data showed that expression of the β-myosin microRNA is induced approximately 100-fold in human diabetic and approximately 300-fold in failing hearts. The increased expression of the β-myosin microRNA in failing hearts compared to a heart from diabetics, showed that the cardiomyopathic alterations in human hearts are correlated with the level of expression of the β-myosin microRNA.

TABLE 1
Relative Expression of the β-Myosin MicroRNA in Diabetic
and Failing Human Hearts.

| | Non- | | | Diabetic/ | |
|---|---|---|---|---|---|
| | failing | Diabetic | Diabetic | Failing | Failing |
| β-myosin | 1 | 41 | 240 | 68 | 78 |
| microRNA | | | | | |
| β-myosin | 1 | 80 | 120 | 280 | 260 |
| microRNA/mir1-1 | | | | | |

### Example 3

### Quantitative Real-Time Analysis of the β-Myosin MicroRNA Using a Stem-Loop Primer

The quantity of β-myosin microRNA can be assessed by using a method based on the current real-time quantitative PCR analysis. This methods uses an approach for cDNA synthesis that does not require ligating an adapter oligonucleotide to the microRNA. A stem-loop primer is used to generate the initial cDNA as described by Chen et al., 2005. Real-time quantification of microRNAs by stem-loop RT-PCR. Nucl Acids Res 33: e179.

What is claimed is:

1. An isolated microRNA selected from the group consisting of SEQ ID NO:1 or the complement of SEQ ID NO:2, SEQ ID NO:3 or SEQ ID NO:4.

2. An isolated microRNA having at least 90% identity to SEQ ID NO:1 or the complement of SEQ ID NO:2, SEQ ID NO:3 or SEQ ID NO:4.

3. A pharmaceutical composition comprising an isolated microRNA selected from the group consisting of SEQ ID NO:1 or the complement of SEQ ID NO:2, SEQ ID NO:3 or SEQ ID NO:4 and a pharmaceutically acceptable carrier.

4. A pharmaceutical composition comprising an isolated microRNA having at least 90% identity to SEQ ID NO:1 or the complement of SEQ ID NO:2, SEQ ID NO:3 or SEQ ID NO:4.

5. A cell comprising an exogenous β-myosin microRNA.

6. A cell comprising an exogenous β-myosin microRNA gene encoding a β-myosin microRNA wherein the β-myosin microRNA is overexpressed or not expressed.

7. The cell of claim 5 or 6, wherein the β-myosin microRNA comprises a nucleic acid sequence selected from the group consisting of SEQ ID NO:1 or the complement of SEQ ID NO:2, SEQ ID NO:3 or SEQ ID NO:4.

8. The cell of claim 5 or 6, wherein the β-myosin microRNA comprises a nucleic acid sequence having at least 90% identity to SEQ ID NO:1 or the complement of SEQ ID NO:2, SEQ ID NO:3 or SEQ ID NO:4.

9. A transgenic animal comprising an exogenous β-myosin microRNA.

10. The transgenic animal of claim 9, wherein the β-myosin microRNA comprises a nucleic acid sequence selected from the group consisting of SEQ ID NO:1 or the complement of SEQ ID NO:2, SEQ ID NO:3 or SEQ ID NO:4.

11. The transgenic animal of claim 9, wherein the β-myosin microRNA comprises a nucleic acid sequence having at least 90% identity to SEQ ID NO:1 or the complement of SEQ ID NO:2, SEQ ID NO:3 or SEQ ID NO:4.

12. The transgenic animal of any one of claims 9 to 11, wherein the β-myosin microRNA is overexpressed.

13. A method for testing a compound for ameliorating a cardiovascular disease, comprising:
(a) providing a cell overexpressing β-myosin microRNA;
(b) contacting the cell with the compound;
(c) and determining whether expression of β-myosin microRNA is reduced.

14. The method of claim 13, wherein the expression of β-myosin microRNA is determined using an assay selected from the group consisting of Northern blot, in situ hybridization, RT-PCR and QT-PCR.

15. A method for testing a compound for ameliorating a cardiovascular disease, comprising:
(a) providing a transgenic animal overexpressing β-myosin microRNA;
(b) administering to the animal the compound;
(c) and determining whether expression of β-myosin microRNA is reduced.

16. The method of claim 15, wherein the expression of β-myosin microRNA is determined using an assay selected from the group consisting of Northern blot, in situ hybridization, RT-PCR and QT-PCR.

17. A method of modulating in a cell expression of a target gene associated with a cardiovascular disease, comprising administering a β-myosin microRNA to the cell.

18. A method of modulating in a subject expression of a target gene associated with a cardiovascular disease, comprising administering a β-myosin microRNA to the subject.

19. The method of claim 17 or 18, wherein the β-myosin microRNA comprises a nucleic acid sequence selected from the group consisting of SEQ ID NO:1 or the complement of SEQ ID NO:2, SEQ ID NO:3 or SEQ ID NO:4.

20. The method of claim 17 or 18, wherein the β-myosin microRNA comprises a nucleic acid sequence having at least 90% identity to SEQ ID NO:1 or the complement of SEQ ID NO:2, SEQ ID NO:3 or SEQ ID NO:4.

21. The method of claim 17 or 18, wherein the β-myosin microRNA is selected from the group consisting of SEQ ID NO:1 or the complement of SEQ ID NO:2, SEQ ID NO:3 or SEQ ID NO:4.

22. A method of determining whether a gene is regulated by β-myosin microRNA comprising;
(a) contacting a cell with β-myosin microRNA and
(b) measuring the expression level of the gene, wherein an increase or descrease in expression level of the gene indicates that the gene is regulated by β-myosin microRNA.

23. The method of claim 22, wherein the expression of β-myosin microRNA is measured using an assay selected from the group consisting of Northern blot, in situ hybridization, RT-PCR and QT-PCR.

24. A method of predicting a subject's risk for developing a cardiovascular disease or disorder, comprising:
(a) obtaining a sample from the subject
(b) determining the level of expression of β-myosin microRNA, wherein an increase in β-myosin microRNA as compared to control indicates the subject is a risk for developing a cardiovascular disease or disorder.

25. The method of claim 24, wherein the step of determining the level of expression of β-myosin microRNA is carried out using an assay selected from the group consisting of Northern blot, in situ hybridization, RT-PCR and QT-PCR.

26. A method of determining whether a compound is effective in ameliorating a cardiovascular disease or disorder, comprising:
(a) providing a transgenic animal overexpressing β-myosin microRNA;
(b) administering to the animal the compound;
(c) and determining whether expression of β-myosin microRNA is reduced, wherein a reduction in β-myosin microRNA indicates that the compound is effective in ameliorating a cardiovascular disease or disorder.

27. The method of claim 26, wherein the step of determining the level of expression of β-myosin microRNA is carried out using an assay selected from the group consisting of Northern blot, in situ hybridization, RT-PCR and QT-PCR.

28. A method of diagnosing a cardiovascular disease or disorder in a subject, comprising:
(a) obtaining a sample from the subject;
(b) determining the level of expression of β-myosin microRNA, wherein an increase in β-myosin microRNA as compared to control indicates the subject has a cardiovascular disease or disorder.

29. The method of claim 28, wherein the step of determining the level of expression of β-myosin microRNA is carried out using an assay selected from the group consisting of Northern blot, in situ hybridization, RT-PCR and QT-PCR.

30. A method of reducing or inhibiting expression of β-myosin microRNA in a cell, comprising contacting the cell with an agent that inhibits β-myosin microRNA expression.

31. The method of claim 30, wherein the cell is a cardiomyocyte or a cardiomyocyte precursor cell.

32. A method of treating a cardiovascular disease or disorder in a subject comprising administering to the subject an agent that inhibits β-myosin microRNA expression.

33. The method of any one of claims 30-32, wherein the agent is selected from the group consisting of an isolated oligonucleotide, a small molecule, an antisense oligonucleotide, and an antagomir.

34. The method of any one of claims 30-32, wherein the agent is an antigomir conjugated to cholesterol.


SEQUENCE LISTING (10 sequences)

SEQ ID NO: 1 - RNA, 21 nt, Oryctolagus cuniculus
  Synthetically generated oligonucleotide
uggaaguggucgucauuguua

SEQ ID NO: 2 - DNA, 21 bp, Homo sapiens
tggaagtggtcgtcattgtta

SEQ ID NO: 3 - DNA, 21 bp, Oryctolagus cuniculus
tggaagtggtcgtcattgtta

SEQ ID NO: 4 - DNA, 21 bp, Mus musculus
tggaagtggtcgtcattgttg

SEQ ID NO: 5 - DNA, 22 bp, Artificial Sequence
  Synthetically generated oligonucleotide
ggcctcccttggctcgttggag

SEQ ID NO: 6 - DNA, 15 bp, Artificial Sequence
  Primer
tactccaacgagcca

SEQ ID NO: 7 - RNA, 15 nt, Artificial Sequence
  Primer
cgagccaagggaggc

SEQ ID NO: 8 - DNA, 20 bp, Artificial Sequence
  Primer
ggaagtggtcgtcattgtta

SEQ ID NO: 9 - DNA, 15 bp, Artificial Sequence
  Primer
ctcaacgagccaagg

SEQ ID NO: 10 - DNA, 23 bp, Artificial Sequence
  Primer
aatggaatgtaaagaagtatgta